(19) United States
(12) Patent Application Publication
(10) Pub. No.: US 2020/0253863 A1
(43) Pub. Date: Aug. 13, 2020

(54) Sterile Buffered Solution Injection to Burn Tissue

(71) Applicant: Vivi Robyn Stafford, Los Angeles, CA (US)

(72) Inventor: Vivi Robyn Stafford, Los Angeles, CA (US)

(21) Appl. No.: 16/271,900

(22) Filed: Feb. 11, 2019

(51) Int. Cl. A61K 9/00; A61K 31/60; A61K 9/08

(52) U.S. Cl. CPC A61K 9/0014; A61K 9/08; A61K 31/60; A61K 9/0019

(57) ABSTRACT

A method for injecting aqueous buffered acid solutions and alkaline solutions into the hypodermis for partial thickness hypodermal burns or to cause denaturation of cells below the dermis.

### BACKGROUND OF THE INVENTION

This invention was not made with U.S. government support.

Methods of regenerating human tissues often comes from modes of injury. Methods of controlled injuries can cause the generation of new tissues in an array of cellular pathways. After the onset of puberty, the dynamics of skin healing and rejuvenating skin properties and the proximate structures of the hypodermis experienced since birth begins to change early in life; meanwhile, the process of skin denaturing continues throughout life. Skin and hair follicles have life spans where old skin cells exfoliate and hair sheds. Over time, the quality of the skin and the hair follicles change. However, through a healthier dermis and hypodermis similarly found in youth can preserve the quality of the skin and the hair and proximate structures. This can be done through regeneration methods. Blood flow is considered essential for life and the micro vessels can become engorged and less effective. Processes of angiogenesis can preserve the health of the dermis and the health of the structures within the hypodermis; such as, hair follicles. Dermal papilla for instance, is known for its support of the hair follicle; however, there are no proven methods in maintaining the health of the dermal papilla. The application of buffered acid solutions and buffered alkaline solutions would and will cause mild irritation to moderate reversible and irreversible damage to cellular matter in the hypodermis. And the dermal papilla which would cause the immune system to upregulate and to heal the dermal papilla preserving the anagen phase of the hair follicle.

Currently there are no approved or rigorously evaluated methods for causing medical grade burns to the hypodermis or the epidermis using injectable methods. Methods have been in place for approaching cell regeneration using laser technology or intense light technology or through methods related to thermoregulation. However, there are no methods to create regeneration of the skin by keeping the epidermis fully intact and by approaching skin regeneration with aqueous buffered acid and buffered alkaline solutions injected into the hypodermis for therapeutic mild to partial thickness burns or denaturing tissues. There have been concerns over skin healing with laser methods which generally are approached on top of the dermis. However, there has been complications from those methods such as blistering of the epidermis and post inflammatory hyperpigmentation. Approaches below the dermis with partial thickness approaches or with denaturing tissue or dissolving cellular damage approaches with the target being the hypodermis eliminates the risks for blistering of the epidermis. The risk of post inflammatory hyperpigmentation is less likely or mitigated and may not approach the occurrences level from any topical approaches.

The demand for buffered acid or alkaline aqueous solutions placed in the hypodermis as a skin corrective measure and for angiogenesis preserving the anagen phase in regards to follicles in the papillary dermis is very high in the cosmetic market and also in those with issue in relations to thinning hair or hair loss. Laser therapy is very expensive with risk of side effects, dermabrasions have a true risk for post inflammatory hyperpigmentation and other untoward events from the current methods in use.

In spite of procedural risks which are low, the growing appeal of cosmetic medical treatments is a testament to the psychological importance of body image and the beneficial outcomes of the procedures as perceived by the populations who seek therapy for their pathology, skin irregularities or for the enhancement they would like to achieve. The significance of a product that delivers outcomes through a subdermal partial thickness burn or reversible or irreversible cellular damage in the hypodermis through buffered aqueous solution rejuvenating the adjacent structures and causing a thicker dermis and healthier proximal structures including angiogenesis with little to no risk to the epidermis or superficial dermis is significant.

### SUMMARY OF THE INVENTION

The present invention is directed to a method for a non-surgical approach and a non-laser or heat modulated approach to causing burns through injections into the hypodermis. The injections would be with aqueous solution consistent with water at 20 to 25 Celsius. The buffered acid solution would range from a pH of greater than 2 to less than 7. The buffered alkali solutions would have a higher pH. After the aqueous acid solutions are injected the structures in the hypodermis will be irritated and reversible and also irreversible cellular damage may occur causing hyperemia. In response to the irritation and injury the hypodermis will trigger a healing response which will sequester cells for regenerating the skin and angiogenesis will also occur. The advantage of this treatment is to cause laser skin rejuvenating results similar to a laser with techniques of injections of aqueous solutions that are hyperemia causing and clinically controlled corrosion in tissues below the dermis. Previously clinical problems are side effects from lasers that approach the skin from the epidermis. These methods are from using a hypodermic needle and applying caustic solutions below the dermis. Through this approach the skin heals deep and there is potentially very little down time with approach being the below the dermis. In addition, this approach causes leaky vessels and allows plasma leakage which means that droplets of autologous blood or exogenous blood can be used for similar results or better results in current platelet rich plasma treatments.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A is a perspective view of a patient before injection of a buffered acid solution.

FIG. 1B is a perspective view of a patient after injection of a buffered acid solution.

FIG. 2A is a perspective view of a patient before injection of a buffered acid solution.

FIG. 2B is a perspective view of a patient after injection of a buffered acid solution.

FIG. 3A is a perspective view of a patient before injection of a buffered acid solution.

FIG. 3B is a perspective view of a patient after injection of a buffered acid solution.

FIG. 4A is a perspective view of a patient before injection of a buffered acid solution.

FIG. 4B is a perspective view of a patient after injection of a buffered acid solution.

FIG. 5A is a perspective view of a patient before injection of a buffered acid solution.

FIG. 5B is a perspective view of a patient after injection of a buffered acid solution.

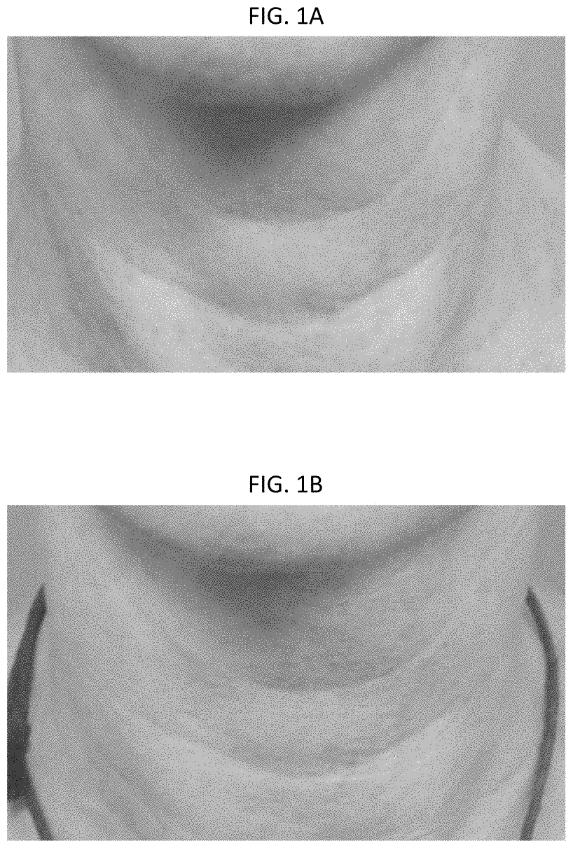

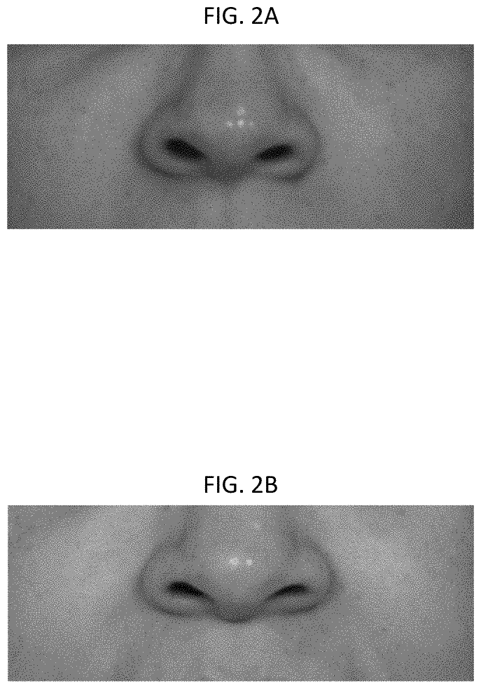

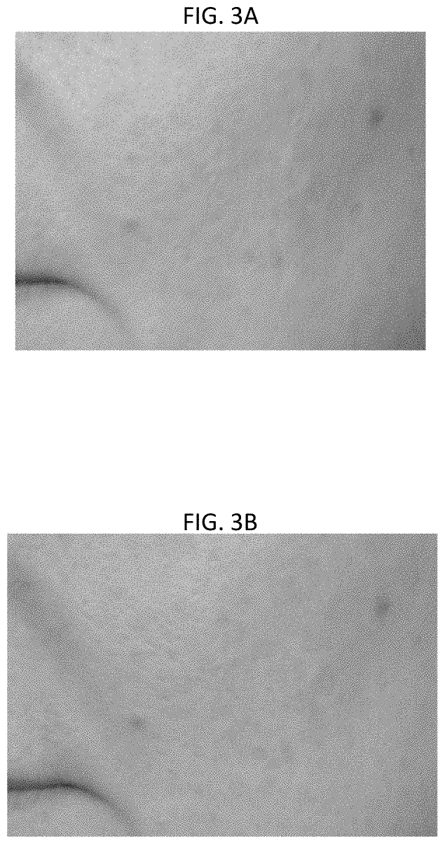

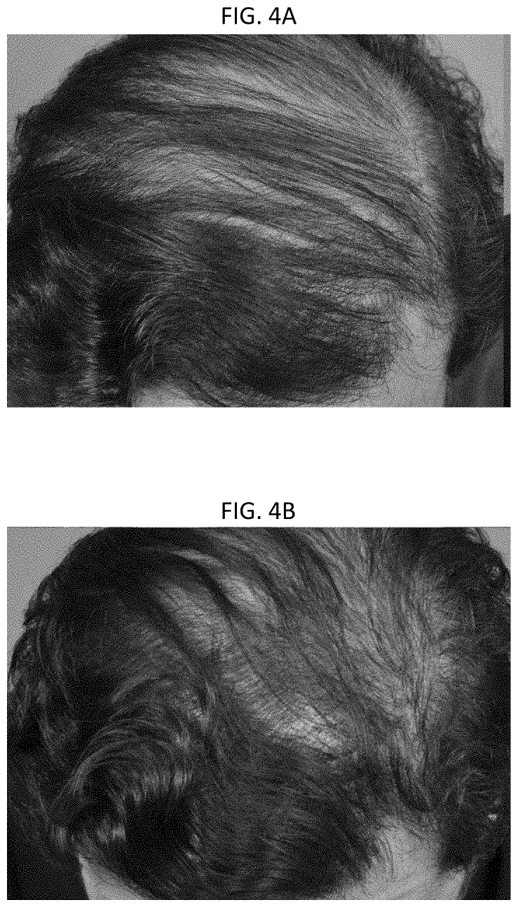

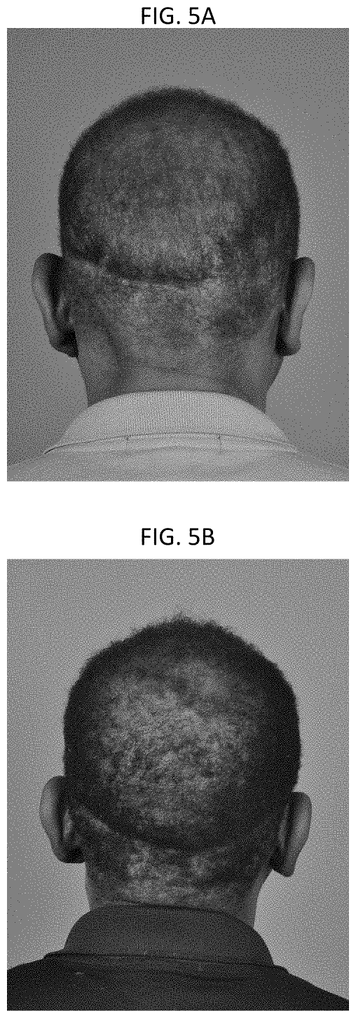

### DETAILED DESCRIPTION OF THE INVENTION

An acid is a substance than neutralizes alkalis. A base is a substance that neutralizes acids. A buffered acid is any substance that is either an acid or base and has had its pH altered either through the addition of an acid or base. There never has been a method for injecting caustic substances into the hypodermis with the purpose of causing a localized burn. Controlled burns have been used in the cosmetic industry with the methods of laser technology and other heated devices. However, there have been no methods for causing localized burns in the hypodermis. Causing localized burns in the hypodermis triggers the body of have a massive cellular response. The massive cellular response forces the hypodermis to repair itself and the papillary dermis. As a result, hair has a health state in the anagen phase and the skin rejuvenates itself giving a clearer complexion from the repair done in the hypodermis. The hypodermis provides health to the skin and the hair and encouraging a healthier hypodermis and could provide for health skin and hair.

A buffered acid solution such as sodium salicylate is injected into the hypodermis at a pH less than 7 and greater than 2 with the goal of causing an irritating burn to the hypodermis and its proximate structures this burn can cause irreversible and revisable damage triggering cellular regeneration and angiogenesis. Additionally, to amplify the response the buffered acid solution may be followed with a buffered alkali solution or vice versa.

The pharmaceutical grade buffered acid solution is mixed in distilled water or a sterile salt water solution and is injected in a 10 cc syringe with an ideal pH of about 5.5. The sterile solution is injected into the hypodermis with a hypodermic needle and the outer layer of the skin is observed. Once the stinging dissipates the treatment may be repeated at that time. For additional response, the treatment may be followed by a nearly neutral solution of an alkaline mixture. The injections are feathered in with hypodermic needles as long as 1½ inch. The solutions contain no micro particles and is the viscosity of water. However polymerized acids may be used to enhance the results. There are currently no skin rejuvenating treatments that rejuvenate the skin through burns from injecting caustic substances. Currently all cosmetic controlled burn treatments that can possibly target tissue below the dermis are with laser technology. This is the only cosmetic treatment designed to burn the tissue through a caustic injury with a needle and a target below the dermis. To mix this solution, a substance such as Dextrose mixed with Salicylic Acid would be buffered to the pH of 5.5, combined with normal saline and injected into the dermis. This mixture may be followed by a sodium bicarb gentle mixture combined in distilled water or normal saline.

Acid solutions, including and not limited to salicylic acid, citric acid, acetylcysteine, calcium lactate, lipoic acid. Malic acid, or gluconic acid are buffered as aqueous solutions towards a pH of 7 or salts thereof. The aqueous solutions are placed in the hypodermis to cause cellular damage by causing chemical burns in the hypodermis and the proximate deep dermis. Such injury will cause a caustic, corrosive, chemical burn where this stimulates the body to repair the injury through the upregulation of macrophages, neutrophils, cytokines, leukotrienes which stimulates angiogenesis and cellular repair. Such effects enhance the growth phase of structures in the hypodermis and its proximate dermis and all surrounding proximate cells, structures, proteins, walls and organelles. The pH of the buffered acid is less than 7 and is greater than 2 with the purpose of causing hypodermal irritation and controlled corrosion.

An acid solution buffered with sodium or calcium carbonate or any buffering agent thereof, which has reducing properties for the purposes of therapeutic benefits; meanwhile, having therapeutic benefits from the controlled, caustic, corrosive and denaturing cellular events in the hypodermis causing reversible and irreversible cellular damage. This damage upregulated in the healing process in the hypodermis and its proximate structures with therapeutic benefits and outcomes.

A medical provider takes the aqueous solution with the viscosity of water. The dynamic viscosity of water is 8.90×10−4 Pa·s or 8.90×10−3 dyn·s/cm2 or 0.890 cP at about 25° C. Meanwhile, water has a viscosity of 0.0091 poise at 25° C., or 1 centipoise at 20° C. The aqueous solution with the viscosity of water is a buffered acid such as; buffered, salicylic with sodium forming sodium salicylate and is injected into the hypodermis with fanning strokes and micro droplets and also with optional hypodermal boluses. Notwithstanding approaches of grid like layering with optional hypodermal boluses. Practitioner may also choose hypodermal boluses. The hypodermal injections are done with 25 to 30-gauge needle attached to 3 cc or 10 cc syringes in an ideal setting.

A method for injecting aqueous buffered acid solutions and alkaline solutions into the hypodermis for partial thickness hypodermal burns or to cause denaturation of cells below the dermis. The partial thickness and superficial hypodermal burns will cause therapeutic injuries. The therapeutic injuries will cause cellular regeneration and angiogenesis. This cellular regeneration and angiogenesis will repair the hypodermis or subcutaneous tissue. The result of the repair of the hypodermis will reduce cellulite, will deter follicles from exiting the anagen phase, will contribute to dermal thickening, will deter melanocyte over activity, will decrease glandular over production of exocrine matter, will repair enlarged micro vessels, will repair pores, will improve skin's color, clarity and tone, will encourage cellular reproduction in the hypodermis, will encourage angiogenesis, will deter the long lasting effects caused by sun damage to the epidermis, will deter the persistence of acne break outs, will tighten pores, will provide enhanced cellular support of the hypodermis and the dermis, will deter hypodermal and dermal dehydration, will diminish the defective cellular structures in scars, will improve the appearance of skin imperfections, will repair the hypodermis while promoting general skin wellness allowing for better results in overall skin health with or without adjunctive therapies. And such solutions for caustic solutions via the buffering of basis with acid solutions or neutral solutions for therapeutic injuries in the hypodermis. The burns will cause hyperemia to more aggressive tissue injuries and denaturation.

I claim:

1. A method of Injecting with a hypodermic needle aqueous solutions for causing burns in human skin below the epidermis and into the hypodermis of the skin specifically through caustic injury to the hypodermis with buffered acid solutions the viscosity of water and buffered alkali solutions. Injections intent to cause a subclinical burn or burns in the subdermis, hypodermis causing localized injury, denaturing tissue and causing hyperemia through the acid or alkali caustic solutions.

2. The method of claim one where Injections of caustic agents causing acid burns of the hypodermis which causes capillary permeability due to the effects of the damage from the acid chemical and where Injections of caustic agents causing alkali burns of the hypodermis which causes capillary permeability due to the effects of the damage from the alkalotic chemical.

3. The method of claim one where Injections of the buffered solution of the acidic origin causes injury resulting in plasma leaks. This plasma leaks encourage angiogenesis and where buffered solution of the alkaline origin causes injury resulting in plasma leaks. This plasma leaks encourage angiogenesis.

4. The method of claim one where Injections of buffered acid solutions causes plasma permeability and also causes the aggregation of fibrin encouraging angiogenesis and where Injections of buffered alkali solutions causes plasma permeability and also causes the aggregation of fibrin encouraging angiogenesis.

5. The method of claim one where Injections of the buffered alkali solutions increases capillary leak ability and plasma permeability allowing for effective droplet treatments of exogenous or autologous plasma or packed cells or red blood and where Injections of the buffered alkali solutions increases capillary leak ability and plasma permeability allowing for effective droplet treatments of exogenous or autologous plasma or packed cells or red blood cells.

6. The method of claim one where Injections of the buffered alkali and buffered acid solutions increases capillary leak ability and plasma permeability for effective treatments with droplets treatments of autologous or exogenous plasma or red blood cells or packed cells.

7. The method of claim one where the subdermal burn is for wound healing purposes, cosmetic purposes including treating discoloration in the skin, hyperpigmentation, hypopigmentation, treating pores, smoothing textures, improving clarity and tone, treating acne and smoothing fine lines and will improve the overall skin health including brightening the skin and decreasing the appearance of cellulite.

8. The method of claim one where the burn is for the purpose enhancing the condition of the subdermal tissues, hypodermal tissues including the subcutaneous tissue by increasing the permeability of plasma and the leak-ability of capillaries for up to 48 hours with subsequent activation of clotting cascade and the pooling of fibrin in clot formation causing the appearance of volumizing the hypodermis and subdermal tissues hence improving the appearance of deep folds, dimples, crevices, folds, wrinkles and enhancing the overall appearance of the three dimensionality of the skin.

9. The method of claim one where the burn is for the purposes of hair growth through the stimulation of angiogenesis and hyperemia.

10. The method of claim one where the subdermal and hypodermal burn is for the purposes of hair growth through the triggering of capillary permeability.

11. The method of claim one where the subdermal and hypodermal burn is for the purposes of hair growth through the enhancement of plasma leak ability, hyperemia and angiogenesis.

12. The method of claim one requires injection methods below the dermis.

13. The method of claim one buffered solutions comprises of solutions with the viscosity of water that are caustic.

14. The method of claim one where the buffered solutions are buffered for tolerability to not cause full thickness injury to the skin.

15. The method of claim one where inflammatory and reactive, proliferative and reparative and maturation and remodeling improves the overall condition and health of the skin without full thickness injury to the dermis.

16. The method of claim one includes buffered acid solutions with the viscosity of water.

17. The method of claim one includes buffered caustic solutions with the viscosity of water without micro particles and is not an injectable implant.

18. The method of claim one includes solutions and polymerized solutions of caustic agents and denatured solutions with the viscosity of water.

19. The method of claim one includes solutions and polymerized solutions of caustic agents, and denaturing solutions with the viscosity of water of salicylic acid, citric acid, dextrose, glycolic, bicarbonate, hyaluronic acid, tartaric acid, malic acid, retinoic acid, phenol, acetic acid, lactic acid, alpha hydroxy acid, calcium carbonate and derivatives thereof.

20. The method of claim one causing hyperemia and angiogenesis and cellular activation in the dermis of the skin containing regenerating the cellular network contained within including the blood vessels, hair follicles, sweat glands, collagen bundles, fibroblasts, nerves and lymph vessels and regenerates the subcutis which contains a network of collagen and fat cells.
